(19) United States
(12) Patent Application Publication
(10) Pub. No.: US 2024/0130750 A1
(43) Pub. Date: Apr. 25, 2024

(54) CLOT ENGAGEMENT AND REMOVAL SYSTEM

(71) Applicant: Neuravi Limited, Galway (IE)

(72) Inventors: Eamon BRADY, Loughrea (IE); Michael GILVARRY, Headford (IE); Mahmood K, RAZAVI, Irvine, CA (US); David VALE, Barna (IE)

(21) Appl. No.: 18/534,117

(22) Filed: Dec. 8, 2023

Related U.S. Application Data
(63) Continuation of application No. 17/671,003, filed on Feb. 14, 2022, now Pat. No. 11,871,949.
Application No. 17/671,003 is a continuation of application No. 16/385,841, filed on Apr. 16, 2019, now Pat. No. 11,246,612.
Application No. 16/385,841 is a continuation of application No. 15/254,133, filed on Sep. 1, 2016, now Pat. No. 10,292,723.
Application No. 15/254,133 is a continuation of application No. 13/823,048, filed on Mar. 13, 2013, now Pat. No. 9,463,036.
(60) Provisional application No. 61/344,848, filed on Oct. 22, 2010.

(51) Int. Cl. A61B 17/221

(52) U.S. Cl. CPC A61B 17/221; A61B 2017/22034

(57) ABSTRACT

A device including a clot engaging device having a collapsed delivery configuration and an expanded deployment configuration. The device can include a plurality of struts defining a plurality of cells; a seam extending along at least a portion of a length of a distal end of the clot engaging device, wherein the seam comprises a pattern configured for gripping a clot and extends between cells of the plurality of cells; and a distal scaffolding region being distally tapered, wherein the distal scaffolding region comprises cells smaller than cells of a remainder of the clot engaging device.

### CROSS-REFERENCE TO RELATED APPLICATIONS

This application is a continuation of U.S. application Ser. No. 17/671,003 filed Feb. 14, 2022, which is a Continuation of U.S. application Ser. No. 16/385,841 filed Apr. 16, 2019, now U.S. Pat. No. 11,246,612, which is a Continuation of U.S. application Ser. No. 15/254,133, filed Sep. 1, 2016, now U.S. Pat. No. 10,292,723, which is a Continuation of U.S. application Ser. No. 13/823,048, filed Mar. 13, 2013, now U.S. Pat. No. 9,463,036, which is a 371 of International Application No. PCT/IE2011/000057, filed Oct. 21, 2011, which claims priority from U.S. Provisional Application No. 61/344,848, filed Oct. 22, 2010, the contents of all of which are incorporated herein by reference in their entireties.

### INTRODUCTION

The invention relates to devices, and methods of removing acute blockages from blood vessels. The invention especially relates to removing acute obstructions from blood vessels. Acute obstructions may include clots, misplaced devices, migrated devices, large emboli and the like. More particularly the invention relates to removing clots from cerebral arteries in patients suffering acute ischemic stroke.

Accessing the neurovascular bed is difficult with conventional technology as the target vessels are small in diameter, are remote relative to the site of insertion and are highly tortuous. Despite the fact that there are over 600,000 acute ischemic strokes in the US each year, clot retrieval devices are used to treat patients in less than <1% of cases. The reasons for this are that conventional technology is either too large in profile, lacks the deliverability to navigate tortuous vessels or is not effective at removing clot when delivered to the target site.

There are significant challenges associated with designing clot removal devices that can deliver high levels of performance. Firstly, there are a number of access challenges that make it difficult to deliver devices. In some patients the configuration of the aortic arch makes it difficult to position a guide catheter in the larger arteries that supply blood to the brain. These difficult arch configurations are classified as either type 2 or type 3 aortic arches with type 3 arches presenting the most difficulty. The tortuosity challenge is even more severe in the arteries approaching the brain. It is not unusual at the distal end of the internal carotid artery that the device will have to navigate a vessel segment with a 180° bend, a 90° bend and a 360° bend in quick succession over a few centimetres of vessel.

Secondly, neurovascular vessels are more fragile than similarly sized vessels in other parts of the body and are in a soft tissue bed. This issue is compounded by the fact that in many instances the clot is firmly wedged in the vessel. More mature and organized clot material is likely to be less compressible than softer fresher clot, and under the action of blood pressure it may distend the compliant vessel in which itis lodged. Thirdly, the clot may comprise any of a range of morphologies and consistencies. In particular long strands of softer clot material may tend to lodge at bifurcations or trifurcations in cerebral vessels, resulting in multiple vessels being simultaneously occluded.

Self-expanding stent-like devices referred to as “stentrievers” are sometimes used to remove clot from cerebral vessels of acute stroke patients. These devices generally pin the clot between the device and vessel wall and embed somewhat into the clot so that the clot can be withdrawn with the device. One disadvantage with this approach is that it relies on pinning the clot between the stentriever and the vessel wall and thus may not restrain the clot effectively when passing a branch vessel or when passing into a vessel that is larger than the fully expanded diameter of the stentriever. Another disadvantage of stentrievers is that they use their radial force to embed in and grip the clot. With soft clot a low level of radial force may be effective, but with firmer clot the level of radial force required to effectively grip the clot may be higher than that which can be safely applied to a cerebral vessel. Therefore stentrievers that have sufficient radial force to deal with all clot types may cause vessel trauma and serious patient injury, and stentrievers that have appropriate radial force to remain atraumatic may not be able to effectively handle all clot types.

The present invention is directed towards providing devices and methods which will address at least some of these issues.

### STATEMENTS OF INVENTION

The invention provides a clot retrieval apparatus which comprises a clot engaging device and a capture basket. The clot engaging device has a collapsed delivery configuration and an expanded deployment configuration, the clot engaging device also has a proximal end and a distal end and an elongate body between the proximal end and the distal end. The capture basket has a collapsed delivery configuration and an expanded deployment configuration, the capture basket also has a proximal mouth which is open when the capture basket is in the deployed configuration. The clot engaging device is axially movable for capture of a clot.

In one aspect the invention provides a clot retrieval apparatus comprising:
a clot engaging device and a capture basket;
the clot engaging device having a collapsed delivery configuration and an expanded deployment configuration, the clot engaging device having a proximal end and a distal end and an elongate body between the proximal end and the distal end, the clot engaging device being connected to a first elongate shaft element;
the capture basket having a collapsed delivery configuration and an expanded deployment configuration, the capture basket having a proximal mouth which is open when the capture basket is in the deployed configuration, the capture basket being connected to a second elongate shaft element, and
the shaft elements being movable relative to one another such that the clot engaging device is axially movable relative to the capture basket for capture of a clot.

In another aspect the invention provides an apparatus for retrieving clot from a blood vessel of a patient comprising:
an elongate shaft, a clot engaging device and a capture basket;
the elongate shaft comprising at least two elongate elements and comprising a distal section and a proximal section, the proximal section extending exterior of the patient, and
the clot engaging device comprising a plurality of struts defining a structure having a collapsed delivery configuration and an expanded deployment configuration, and having a proximal end and a distal end, and being attached at its proximal end to the distal section of a first elongate element of the shaft;
the capture basket comprising a structure with a capture net and an inlet mouth and having a collapsed delivery configuration and an expanded deployment configuration, and being attached at its proximal end to the distal section of a second elongate element of the shaft;
the capture basket being axially movable relative to the clot engaging device to pin a clot between the two structures.

In one embodiment at least the distal end of the clot engaging device is movable to enter the proximal mouth of the capture basket for capture of a clot.

In one case the elongate body of the clot engaging device comprises a distal section, a proximal section and an intermediate section between the proximal and the distal sections.

In one embodiment in the deployed configuration, the distal section of the clot engaging device has a diameter which is smaller than a diameter of the intermediate section.

In one case in the deployed configuration, the proximal section of the clot engaging device has a diameter which is smaller than a diameter of the intermediate section.

In the deployed configuration, at least the intermediate section of the clot engaging device has a generally tubular shape.

In one embodiment the clot engaging device comprises a mesh.

The mesh may comprise a plurality of struts. At least some of the struts may form closed cells.

In one embodiment the clot engaging device defines a pathway for the second shaft element.

In one case the capture basket comprises a capture net and a support frame for the capture net.

The support frame may be connected to the second shaft element.

In one embodiment the clot retrieval apparatus comprises a control element which extends proximally from the support frame of the capture basket for operation by a user. The control element may comprise a tether for controlling the operation of the frame.

The invention also provides various methods for retrieving a clot from a blood vessel of a patient.

In one aspect the clot retrieval method comprises the steps of:
providing a clot retrieval apparatus comprising an engaging device and a capture basket;
crossing a clot with a microcatheter;
advancing the clot retrieval apparatus through the microcatheter and across the clot;
deploying the capture basket distal of the clot;
deploying the clot engaging device within the clot at an initial deployment site;
retracting the clot engaging device and the capture basket together to a location proximal of the initial deployment site;
retracting the capture basket towards the clot engaging device to capture the clot therebetween; and
withdrawing the clot engaging device, the capture basket and the captured clot proximally.

### BRIEF DESCRIPTION OF THE DRAWINGS

The invention will be more clearly understood from the following description of some embodiments thereof, given by way of example only, with reference to the accompanying drawings, in which:

FIG. 1 is a side view of a device according to the invention for removing an obstruction to a vessel;

FIG. 2 is a side view of a similar device for removing an obstruction to a vessel;

FIG. 3 illustrates the device of FIG. 2 with a distal basket retracted relative to a clot engaging section;

FIGS. 4 and 5 are side views of the basket of the device in the collapsed and expanded configurations respectively;

FIG. 6 is a side view of a frame in a figure eight pattern;

FIG. 7 is an isometric view of a clot aging portion of a device of the invention; and

FIGS. 8 to 14 illustrate one method of use of a device of the invention.

### DETAILED DESCRIPTION

The invention provides an apparatus and methods for the removal of obstructions in vessels. In particular, the invention is directed towards the treatment of occlusions to blood vessels, especially arterial vessels, and more particularly the removal of occlusive clots from cerebral arterial vessels.

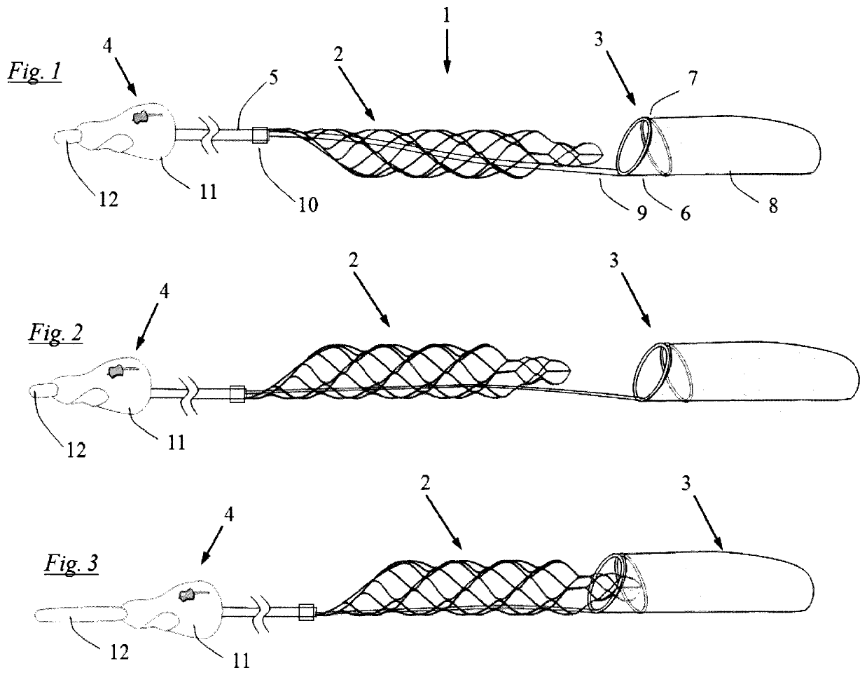

With reference to FIG. 1 there is shown a schematic representation of a device 1 according to the invention for the removal of an obstruction to a vessel. The device 1 comprises a clot engaging device 2 and a capture basket 3. The clot engager 2 has a collapsed state for delivery through the vasculature and an expanded state for engagement with the clot and for disengaging the clot from the vessel wall. The clot engager 2 is connected to a first elongate shaft element which in this case is provided by a support shaft 5 by means of collar 10 or by any other suitable joining method. The support shaft 5 may be connected at its proximal end to a first element 11 of a handle 4. The capture basket 3 has a collapsed configuration for delivery and an expanded configuration for clot engagement and capture. The capture basket 3 comprises a frame 7, a tether 6, and a capture net 8. The frame 7 is connected to a second elongate shaft element which in this case is provided by a support shaft 9. The shaft 9 may be connected at its proximal end to a second element 12 of the handle 4.

The device is configured so that relative movement may be effected between the clot engager 2 and the capture basket 3. In the embodiment shown this movement may be effected by relative movement of components 11 and 12 of the handle 4 which are connected to the shaft elements 5, 9. In another embodiment the handle 4 is detachable from the shaft and in yet another embodiment the device is configured without any handle.

In one embodiment the device is configured so that at least a distal portion of the clot engager 2 can enter the capture basket 3. The degree to which the clot engager can enter the basket 3 may be controlled by limiting the travel of the shaft 9 relative to the shaft 5. The limitation may be provided for example by a stop on the shaft 9 as the clot engager 2 is connected to the shaft 5 and the basket is connected to the shaft 9.

In one case the clot engager 2 comprises multiple struts. The struts may form at least one closed cell. The shaft 9 may pass through at least one of the closed cells. In another embodiment the clot engager 2 comprises multiple struts, forming multiple cells and forming a generally tubular shape, with at least one open seam along the length of the engager 2, and with the shaft 9 passing through one of said seams. In one embodiment the shaft 9 is positioned partially within and partially outside of the clot engager 2 in the expanded configuration and fully within the clot engager in the wrapped configuration, passing from with into without via a pathway defined by a seam extending to the distal end of the device.

FIG. 2 illustrates a device similar to FIG. 1 in which like parts were assigned the same reference numerals. In this case the clot engager 2 is positioned proximal of the capture basket 3. FIG. 3 shows the device of FIG. 2 in a configuration in which the capture basket 3 has been retracted over the distal end of the clot engager 2 by movement of the second handle element 12 relative to the first handle element 11.

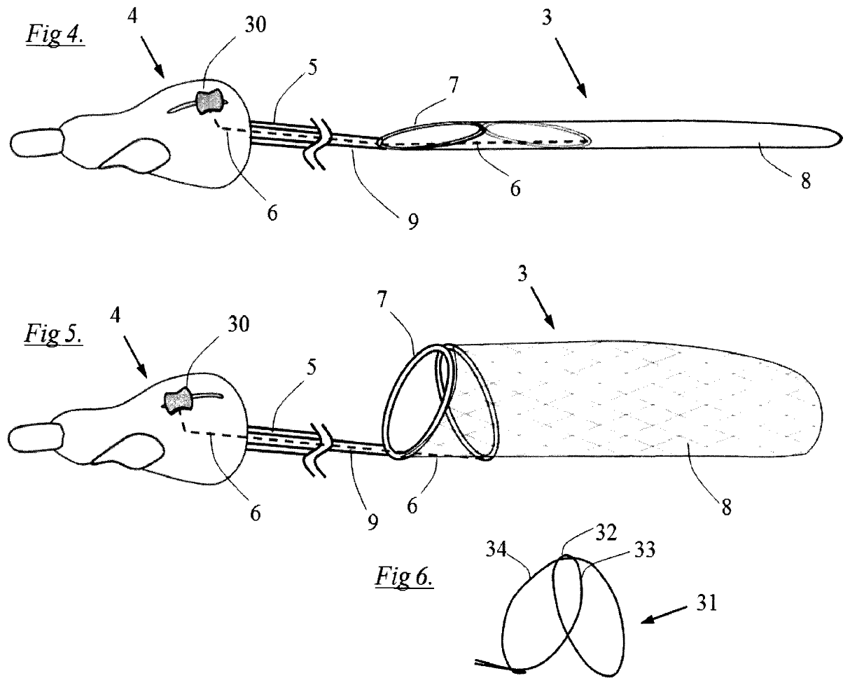

FIGS. 4 and 5 are side views of the proximal and distal ends of a device of the invention in which a clot engager portion is omitted. In the embodiment illustrated the capture basket 3 has a control tether 6 that can be tensioned to control the expansion of the frame 7 of the clot basket 3 or to increase the radial force of the frame 7 of the clot basket 3. The user can control the diameter to which the frame 7 expands by controlling the position of a movable control element such as a button 30 on the handle 4. FIG. 4 shows the control button 30 in an advanced position with the basket 3 in a collapsed configuration. This collapsed configuration of the device facilitates loading and advancement through a microcatheter. In this configuration the frame 7 has a low expansion force and is thus very flexible and easy to deliver.

FIG. 5 shows the frame 7 in an expanded configuration. In this configuration the control button 30 is in a retracted position to tension and withdraw the tether 6 and thus expand and strengthen the frame 7. In one embodiment the frame 7 and the basket 3 can be set to different diameters by adjusting the position of the button 30.

FIG. 6 illustrates one embodiment of a frame 31 in which the frame 31 is formed in a figure of eight pattern so that frame struts 33 and 34 at crossover point 32 can move relative to each other to facilitate the adjustment of the frame to different vessel diameters.

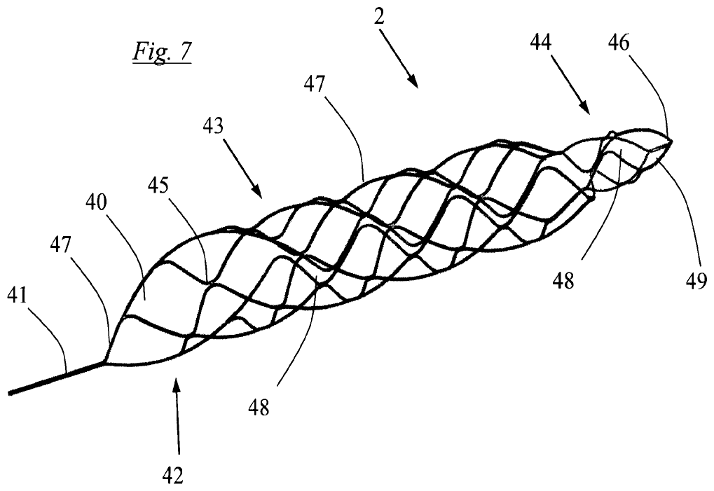

FIG. 7 is an isometric view of a clot engagement device 2 of the invention. The clot engagement device 2 has a proximal end 41 and a distal end 46, and between these ends there are a proximal section 42, amid section 43 and a distal section 44. Multiple struts 47 connect one end of the device to the other. In certain sections of the device the struts 47 are configured to form cells 40 with connecting apices 45. In one case (as shown in FIG. 7) the distal section 44 has a smaller diameter than the mid-section 43, so that the distal section may enter the mouth of a basket or capture net (not shown). In one case (as shown in FIG. 7) the cells 40 are not fully connected around the device circumference at all points along the length of the device, effectively leaving an open seam 48 extending from the proximal end to distal end. This seam provides a pathway through which an elongate shaft entering the device through the open mouth of section 40 may exit the middle section 43 or distal section 44 of the device, such as shown in FIGS. 1, 2 and 3. Thus the distal end of device 2 may enter the mouth of a basket attached to the end of such a shaft, such as illustrated in FIG. 3 by way of example. In another embodiment the seam may run only partially along the length of the device, spanning at least the distal section 44 where it provides an exit pathway for the elongate shaft. This seam may run axially along the device as shown or may run in a more helical-like pattern around the device, or may be staggered and discontinuous, said seam patterns providing additional engagement features to aid in gripping of the target clot. The distal end 46 is in this case configured to enter capture basket 3 of the device of FIG. 1. Distal struts 49 act as guiderails in the embodiment shown to facilitate this entry by providing a smooth and snag free surface to contact and pass through the inlet mouth 34 of the frame 31 in FIG. 6 of a capture basket.

The material of the device may be nitinol or another super elastic or shape memory material, and may be formed from multiple wires or may be cut from a tube or a flat sheet, and may be heat set to define a preferred expanded geometry.

FIGS. 8 to 14 show a series of procedural steps associated with using the device 1. The standard steps involved to place a guide or sheath access catheter and cross the clot with a guidewire and microcatheter are not shown, but will be easily understood by one skilled in the art.

FIG. 8 shows a clot 51 lodged in a bifurcation with vasculature 50.

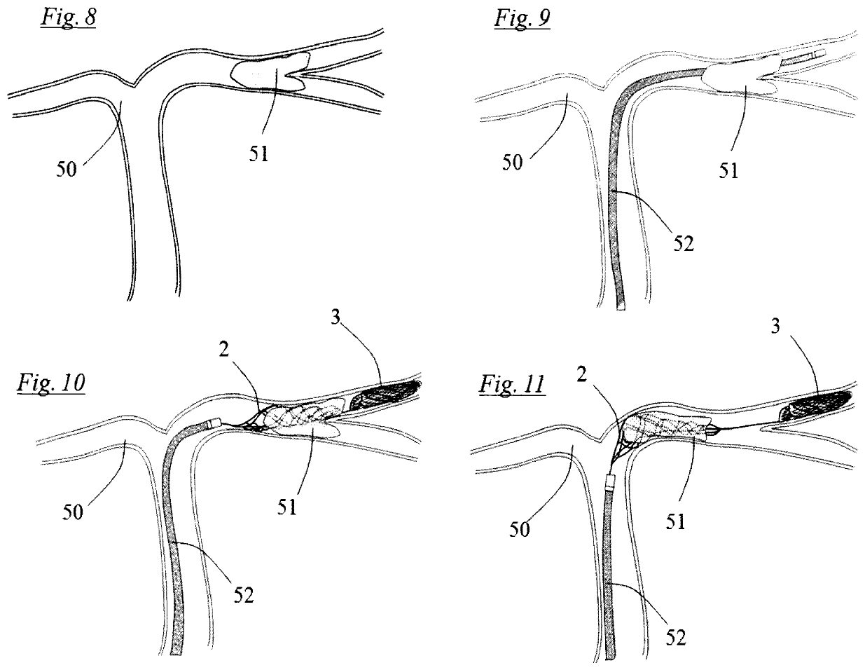

FIG. 9 shows a microcatheter 52 that has been advanced across the clot 51, typically with the aid of a guidewire (not shown) which is then withdrawn to leave the lumen of the microcatheter free for the advancement of a clot retrieval device.

The clot retrieval device is then loaded into the proximal end of the microcatheter 52 and advanced to a target deployment site with the aid of fluoroscopy. The device is positioned so that the capture basket 3 is distal of the clot 51 and at least a portion of the clot engager 2 is within the clot. The microcatheter 52 is then retracted allowing the devices to deploy and expand as shown in FIG. 10. The clot engager 2 expands and engages with body of the clot 51. If the capture basket 3 employs a tether activation system as described above the tether may be tensioned to effect deployment of the capture basket 3 or to increase the opening force so that the basket can engage and encapsulate clot without collapse.

The clot engager 2 is then retracted, pulling the clot 51 out of the bifurcation and into a more proximal section of the vasculature as shown in FIG. 11.

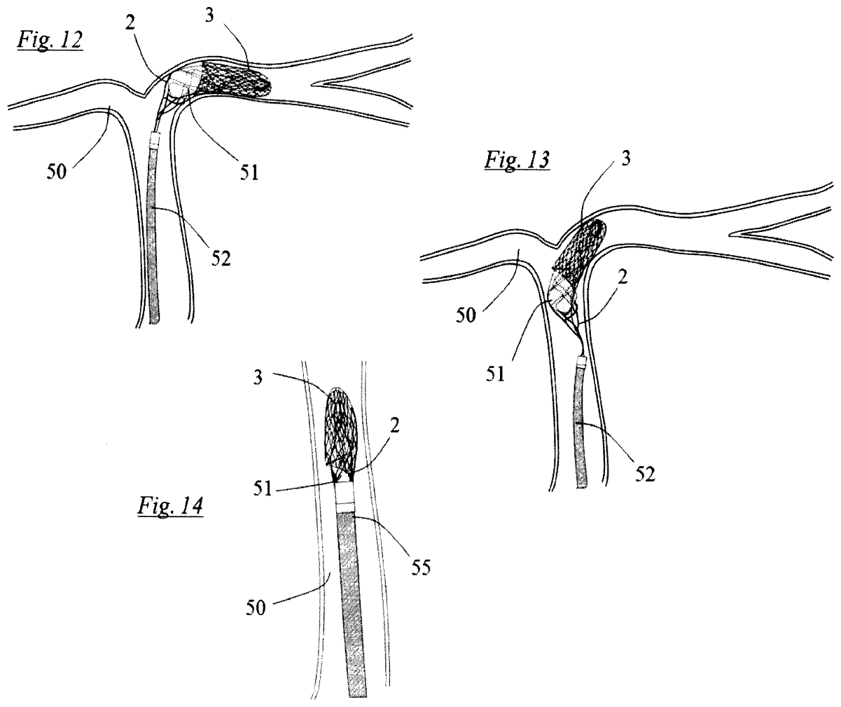

The capture basket 3 can then be retracted to encapsulate some or all of the clot 51 and the clot engager 2 and to pin the clot 51 between the clot engager 2 and the capture basket 3 as shown in FIG. 12.

The clot engager 2, capture basket 3 and captured clot 51 can then be safely withdrawn proximally with clot held securely as the system passes any branch vessels and into larger proximal vasculature as shown in FIG. 13.

FIG. 14 shows the device and clot being withdrawn into a guide catheter or sheath 55.

Removal of the device and captured clot may be assisted by the use of a distal access catheter or similar device with or without the use of aspiration. A flow obstructing cuff on the distal end of a guide catheter or sheath may also be employed to aid in the effectiveness of the use of aspiration while extracting the device and captured clot from the patient.

An alternative sequence of steps that may be used with the device of this invention would involve:
crossing the clot with a microcatheter as described above;
advancing the clot retrieval device through the microcatheter and across the clot;
withdrawing the microcatheter to a location proximal of the clot to leave the capture basket deployed distal of the clot and the clot engager deployed within the clot;
retracting the clot engager and capture basket together to a location proximal of the initial deployment site;
retracting the capture basket a further distance towards and over the clot engager: and
withdrawing the clot engager, the capture basket and the captured clot proximally out of the patient.

Another of the many ways in which a device of this invention may be employed involves the use of a variant of the device without a handle or with a detachable handle. Such a device may be deployed across the clot as described in one of the methods above, after which the basket may be left in place protecting the distal vessel bed while the clot engager is advanced and withdrawn multiple times to remove a clot. This system has the advantage that the basket shaft acts a guide rail to facilitate rapid re-advancement of the clot engager to the target site on each pass. An extension wire may be added to the proximal end of the basket shaft 9 to enable the user to maintain good control of the basket while removing the clot engager from the patient between passes. Alternatively the clot engager 2 and its shaft 5 may be configured in the manner of a rapid exchange catheter so that they can be advanced and retracted over the basket shaft 9 without the need for any extension wire.

It will be understood from all of the above that this device has features that enable it to be used in a variety of different ways to retrieve obstructions from vessel, so that the precise method of use can be tailored to suit the specific needs of any given situation. The clot engager is designed to engage with clot by means of the engager struts, and more specifically the distal apices of the engager cells, embedding in and snagging the clot. Expansion of the engager also pins the clot between the engager and the vessel wall and assists this embedding process. The basket frame 7 is designed to engage the clot from its distal end and assists in guiding the capture net 8 over the clot. The frame 7 and clot engager 2 can also be used to pin the clot between these two elements of the device. This is of particular advantage in ensuring that a firm grip is held on the clot as it is withdrawn against the flow past branch vessels and into larger more proximal vasculature. The clot engager may also be employed to constrain the clot while the basket is withdrawn over the clot. This may be of particular advantage in cases where the clot is firmly lodged in the vessel and a significant force is required to disengage and remove it. Using the clot engager in a compressive mode while retracting the basket over the clot shields the distal vasculature from potentially traumatic tensile forces that would otherwise be exerted on them.

Accessing cerebral vessels involves the use of a number of commercially available products and conventional procedural steps. Access products such as guidewires, guide catheters and microcatheters are known and are regularly used in procedures carried out in cerebral vessels. Such systems are described for example in WO2010/010545A and US20110160763A the entire contents of which are incorporated herein by reference.

Modifications and additions can be made to the embodiments of the invention described herein without departing from the scope of the invention. For example, while the embodiments described herein refer to particular features, the invention includes embodiments having different combinations of features. The invention also includes embodiments that do not include all of the specific features described.

The invention is not limited to the embodiments hereinbefore described which may be varied in construction and detail.

What is claimed is:

1. A device, comprising:
a clot engaging device having a collapsed delivery configuration and an expanded deployment configuration, the clot engaging device comprising:
a plurality of struts defining a plurality of cells;
a seam extending along at least a portion of a length of a distal end of the clot engaging device; and
a distal scaffolding region being distally tapered,
wherein the seam extends helically about a circumference of at least a portion of the clot engaging device.

2. The device of claim 1, wherein the seam comprises a pattern configured for gripping a clot and extends between cells of the plurality of cells.

3. The device of claim 1, wherein the distal scaffolding region comprises cells smaller than cells of a remainder of the clot engaging device.

4. The device of claim 1, wherein the seam extends circumferentially about a portion of the clot engaging device.

5. The device of claim 1, wherein the seam initiates at a location distal of a proximal end of the clot engaging device.

6. The device of claim 1, wherein the seam extends through the distal end of the clot engaging device.

7. The device of claim 1, wherein the clot engaging device is self-expandable.

8. The device of claim 1, wherein the clot engaging device includes a proximal end eccentrically attached to a distal end of a shaft.

9. The device of claim 1, wherein the device further comprises a capture basket disposed distal to the clot engaging device.

10. The device of claim 9, wherein at least a portion of the distal scaffolding region is configured to enter at least a portion of the capture basket.

11. The device of claim 9, wherein the capture basket is configured to transition between a collapsed configuration and an expanded configuration.

12. A device, comprising:
a clot engaging device having a collapsed delivery configuration and an expanded deployment configuration, the clot engaging device including:
a plurality of struts defining a first plurality of cells of a first size;
a seam extending along at least a distal portion of the clot engaging device; and
a distal scaffolding region comprising a second plurality of cells of a second size, the second size being smaller than the first size,
wherein the seam extends circumferentially about a portion of the clot engaging device.

13. The device of claim 12, wherein the seam is configured for gripping a clot and extends between cells of the plurality of cells circumferentially about at least a portion of the clot engaging device.

14. The device of claim 12, wherein the seam extends helically about an entirety of a circumference of the clot engaging device.

15. The device of claim 12, wherein the seam initiates at a location distal of a proximal end of the clot engaging device.

16. The device of claim 12, wherein the seam extends along an entire length of the clot engaging device.

17. The device of claim 12, wherein the clot engaging device includes a proximal end formed with a distal end of a shaft.

18. The device of claim 17, wherein the clot engaging device is eccentric to the shaft.

19. The device of claim 12, wherein a proximal end of the clot engaging device includes an open proximal mouth that extends in a plane angled with respect to a longitudinal axis of the clot engaging device.
